(19) United States
(12) Patent Application Publication
(10) Pub. No.: US 2002/0107183 A1
(43) Pub. Date: Aug. 8, 2002

(54) Pharmaceutical preparations comprising cyclosporin for oral administration

(76) Inventors: Monika Petszulat, Laupheim (DE); Klaus Neuer, Schwendi (DE); Hatto Walch, Laupheim (DE)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 10/067,702

(22) Filed: Feb. 5, 2002

Related U.S. Application Data
(63) Continuation of application No. 09/637,260, filed on Aug. 11, 2000.
Application No. 09/637,260 is a continuation of application No. 09/229,243, filed on Jan. 12, 1999, now abandoned.
Application No. 09/229,243 is a continuation of application No. 08/537,683, filed on Jan. 22, 1996, now abandoned.
Application No. 08/537,683 is a 371 of international application No. PCT/EP94/01228, filed on Apr. 20, 1994.

(30) Foreign Application Priority Data
Apr. 20, 1993 (DE) P4312728.2
Apr. 8, 1994 (DE) P4412201.2

(51) Int. Cl. A61K 9/14; A61K 38/13

(52) U.S. Cl. 514/9; 424/486

(57) ABSTRACT

The invention relates to new cyclosporin-comprising oral pharmaceutical preparations. The new pharmaceutical preparations can be produced more easily and have a good bio-availability. In addition to cyclosporin as active ingredient the preparations contain an alkylene-polyether or alkylene-polyester. Optionally, an alkylene-polyole, an alkylene-glycole, a polyalkylene-glycole, an alkyldiether or partial ether of a lower monooxyalkandiole or polyoxyalkandiole and/or a vegetable oil or its hydrated or hydrolysed product may be contained.

The invention relates to new pharmaceutical preparations comprising cyclosporin as active ingredient for oral administration.

Cyclosporins are a class of peptides which are used as immunosuppressants, in particular. Moreover, cyclosporins are known to have antiphlogistic and antiparasitic effects. Therefore, the use of cyclosporins is not limited to immunosuppressants only but relates to all phlogistic diseases including various auto-immune diseases as well as other phlogistic conditions, in particular, phlogistic conditions in which auto-immune processes play a role. The above phlogistic conditions also include, in particular, arthritic diseases such as rheumatoid arthritis as well as rheumatic diseases. Cyclosporins can be used as antiparasitic agents e.g. for the treatment of protozoal infections such as malaria.

Cyclosporins are highly hydrophobic substances, having the consequence that it is difficult to easily process them into pharmaceutical preparations ensuring further sufficient bio-availability. The latter aspect is particularly important, because the cyclosporins possess nephrotoxic side-effects of essential importance. Cyclosporin-containing pharmaceutical preparations proposed so far are based on the use of an alcohol and/or oils or similar vehicles in connection with a surface-active agent. Such preparations are known from DE-OS 29 07 460, for instance. The use of such liquid compositions, however, is accompanied by a number of disadvantages and difficulties. The use of oils or comparable vehicles on oil basis leads to impairment of the sense of taste, in particular, in the case of long-time administration as a consequence of long-term therapy. Since for dissolving the active ingredient a high amount of alcohol is required, the result will be that in addition the patient is permanently administered alcohol and in the case of evaporation of the alcohol during long-term use the active ingredient precipitates. The attempt to offer such preparations in the form of soft gelatin capsules did not yield a satisfactory solution either due to the higher expenditure connected therewith.

DE-OS 40 03 844 proposes a preparation system which in addition to the active ingredient contains a fatty acid saccharide monoester and a diluent or vehicle by means of which it is said to be possible to provide solid, semi-solid and liquid preparations having a content of cyclosporin in a sufficiently high concentration, so that thus oral administration is comfortably possible and an improved efficiency, for instance, with respect to the bio-availability properties will be achieved. Accordingly, these forms of administration contain at least two components in addition to the active ingredient.

The applicant now has surprisingly found a preparation system for oral administration by means of which it is possible to provide a cyclosporin-comprising pharmaceutical preparation for oral administration, which in addition to the active ingredient cyclosporin contains only one vehicle component. Said component is an alkylene-polyether or alkylene-polyester or any mixture thereof, in which the vehicle system must have an HLB of at least 10. The preparations according to the invention yield a bio-availability of the active ingredient which at least is comparable with the best known cyclosporin-containing preparations.

Having a comparably good bio-availability the pharmaceutical preparations according to the invention can be produced in a more economical way, avoid additives impairing the sense of taste as well as the disadvantageous alcohol contained and, in addition, lead to a better patient compliance within the sense that the total weight of the formulation to be administered is reduced as compared with known preparations, with the active ingredient concentration staying the same.

Therefore, the invention relates to pharmaceutical preparations for oral administration, containing cyclosporin as active ingredient and being composed as follows:

a) a cyclosporin as active ingredient,

b) an alkylene polyether or alkylene-polyester as vehicle or any mixture thereof, with the HLB being at least 10.

Optionally, the preparations according to the invention may contain as further component (c) an alkylene polyole, an alkylene glycole, a polyalkylene glycole, a C₂₋₅-alkyldiether or partial ether of a lower monooxyalkandiole or polyoxyalkandiole having 2 to 15 carbon atoms and/or a vegetable oil or its hydrated or hydrolysed product.

Moreover, the preparations according to the invention can contain further known, common and pharmaceutically acceptable additives (d) such as are known in the field of the production of oral formulations.

In parts by weight the preparations according to the invention contain 1 to 50 parts by weight of (b) and/or 0.5 to 20 parts by weight of (c) per part by weight active ingredient, preferably 5 to 10 parts by weight (b) and/or 1 to 10 parts by weight (c) per 1 part active ingredient and, in particular 5 parts by weight (b) and/or 1 part by weight (c) per 1 part by weight active ingredient.

In the case of component (b) it suitably pertains to C₃ to C₅ alkylene-triolether or C₃ to C₅ alkylene-tricester, in particular glycerine. These also include e.g. transesterification products of the alkylene-triolesters with other monooles, dioles or polyoles as well as those substances described under “component C⁵” in DE-OS 40 03 844. Saturated polyglycolised glycerides having an HLB of at least 10 are particularly advantageous. Preferably, the saturated, polyglycolised glycerides known under the mark term Gelucire (the term Gelucire is a trademark of the company Gattefossé) are used and, in particular, the Gelucire^{R} 35/10, 44/14, 42/12, 50/13, 53/10 and any mixtures thereof, in which connection the HLB of the vehicle components used is at least 10.

The optional component (c) comprises, for instance, diethers or partial ethers of lower (C₂₋₁₂) monooxyalkandioles or polyoxyalkandioles such as are described in DE-OS 39 30 928 in the section relating to the component 1.1. The optional component (c) further comprises C₃₋₅ alkylene polyoles, C₂₋₄ alkylene glycoles, poly-(C₂₋₄-alkylene)-glycoles, and vegetable oils as well as their hydration and/or hydrolysis products such as castor oil, olive oil, palm oil, coconut oil, corn oil, sesame oil. The component (c) may be contained as single substance or in any mixtures. Preferred examples of the component (c) are glycerine, propylene glycole and polyalkylene glycole having a molecular weight of up to 600, in particular transcutol and castor oil and the hydrated and hydrolysed products thereof.

The further usable additives pertain to pharmaceutically acceptable additives common in the field of oral forms of administration. Examples thereof are the release of controlling substances, thickening agents, preservatives, stabilizers, flavorings, binding agents, lubricants and the like. These additives may amount to up to 50% of the total composition, however, preferably does not exceed 25% and, in particular, not 10% of the total composition.

All of the known natural and synthetic cyclosporins including their analogs and derivatives are suitable for the use in preparations according to the invention. Examples of such cyclosporins are found e.g. in DE-OS 40 03 844 and DE-OS 40 05 190. Preferably cyclosporin A is used.

The oral forms of administration include e.g. liquids, granulates and solid forms such as tablets and capsules which can be produced according to the common methods known to the person skilled in the art.

The oral forms of administration according to the invention usually are available in standard dose form and contain about 20 to 200 mg, preferably 50 to 100 mg active ingredient per standard dose.

The following examples serve the further illustration of the invention.

### EXAMPLES

| | Components | Amount (mg) |
|---|---|---|
| 1. | Cyclosporin A | 50.0 |
| | Gelucir 53/10 | 300.0 |
| | Total | 350.0 |
| 2. | Cyclosporin A | 50.0 |
| | Gelucir 44/14 | 250.0 |
| | Propylene glycole | 50.0 |
| | Total | 350.0 |
| 3. | Cyclosporin A | 50.0 |
| | Gelucir 50/13 | 250.0 |
| | Transcutol | 75.0 |
| | Total | 375.0 |
| 4. | Cyclosporin A | 50.0 |
| | Gelucir 44/14 | 250.0 |
| | Total | 300.0 |
| 5. | Cyclosporin A | 50.0 |
| | Gelucir 50/13 | 250.0 |
| | Propylene glycole | 50.0 |
| | Total | 350.0 |
| 6. | Cyclosporin A | 50.0 |
| | Gelucir 35/10 | 250.0 |
| | Propylene glycole | 25.0 |
| | Total | 325.0 |
| 7. | Cyclosporin A | 50.0 |
| | Gelucir 53/10, | 275.0 |
| | 42/12 | |
| | Transcutol | 50.0 |
| | Total | 375.0 |
| 8. | Cyclosporin A | 50.0 |
| | Gelucir 42/12 | 300.0 |
| | Glycerine | 25.0 |
| | Total | 375.0 |
| 9. | Cyclosporin A | 50.0 |
| | Gelucir 50/13 | 250.0 |
| | Castor oil | 75.0 |
| | Total | 375.0 |

Production: The compositions of examples 1 to 9 are produced in that the component (b) is melted by heating preferably to at least 60° C. and the active ingredient (a) is dissolved therein by stirring. If desired, optional component (c) is added to the melted mass.

Subsequently, the preparations obtained are filled, for instance, in liquid form into hard-gelatin capsules of the desired size in the concentrations desired. The compositions can also be further processed to tablets in the known manner. For this purpose, the melted masses are produced as described in the above. The liquid melted masses are poured out and after solidification diminuited by means of a sieving machine. The granulates produced such are mixed with the usual adjuvants such as slip agents and lubricants, blasting agents, fillers, flavor corrigents etc. The finished mixtures are pressed to tablets having the desired content of cyclosporin. The tablets may also be coated with a protective cover.

Bio-Availability:

Examinations as to the bio-availability of the compositions according to the invention on dogs.

A group of six Beagle dogs was used for the bio-availability examinations. The test drugs were orally applied to the animal with an empty stomach by means of oesophageal sounds. At defined times blood is taken from the Vena saphena of the animals and collected in corresponding plastic tubes with EDTA additive. The blood samples are stored until assaying at −18° C. Assay of the cyclosporin takes place in the whole blood by means of fluorescence-polarisation immunoassay (FPIA).

The areas under the curves (AUC) in which the blood drug concentration is applied relative to time were calculated according to the trapezoid rule. The average AUC values of compositions according to the invention are shown in the following table in comparison to the commercially available substances of cyclosporin drinking solution and cyclosporin capsules (Sandimmun^{R}) which were ascertained in the same manner in the same dosage with the same dogs.

| Examples | AUC (0-12 h) ng/ml |
|---|---|
| Example 2 | 9035 + 2134 |
| Example 3 | 7859 + 1512 |
| Example 4 | 7552 + 1194 |
| Example 5 | 8228 + 857 |
| Cyclosporin Drinking Solution | 7980 + 1320 |
| Cyclosporin Capsules | 8098 + 1504 |

As the above tests on the bio-availability show, the pharmaceutical preparations according to the invention make it possible to provide the active ingredient cyclosporin in such an oral form that its bio-availability at least corresponds to the preparations known so far.

1. Pharmaceutical preparation for oral administration containing as the only component or consisting of
(a) a cyclosporin as active ingredient, and
(b) an alkylene-polyether or alkylene-polyester either alone or in any mixture as vehicle, whereby the HLB of the component (b) used being at least 10.

2. Pharmaceutical preparation according to claim 1, further containing (c) an alkylene-polyole, alkylene-glycole, a polyalkylene-glycole, an alkyldiether or partial ether of a lower monooxyalkandiole or polyoxyalkandiole and/or a vegetable oil or its hydrated or hydrolysed product either alone or in any mixtures.

3. Preparation according to claim 1 or 2, in which the respective components (a), (b) and/or (c) are available in the following weight ratios: 1:1-50:0.5-20, preferably 1:5-10:1-10, in particular 1:5:1.

4. Preparation according to one of claims 1 to 3, in which the component (b) is chosen from among saturated polyglycolised glycerides.

5. Pharmaceutical preparation according to the previous claim 4, in which the component (b) is chosen from among the Gelucires Gelucir^{R} 35/10, 44/14, 42/12, 50/13, 53/10 and any mixtures thereof.

6. Pharmaceutical preparation according to claim 2, in which the additional component (c) is chosen from among glycerine, propylene glycole, PEG with MG up to approx. 600, transcutol and castor oil.

7. Pharmaceutical preparation according to one of the previous claims in the form of hard-gelatin capsules or in the form of a tablet.

8. Pharmaceutical preparation according to one of the previous claims, characterized in that the active ingredient concentration is 20 to 200 mg, preferably 50 to 100 mg per dose unit.
